(19) United States
(12) Patent Application Publication
(10) Pub. No.: US 2012/0226082 A1
(43) Pub. Date: Sep. 6, 2012

(54) METHOD OF PRODUCING STRAIGHT-CHAIN SATURATED HYDROCARBON IN DIRECT PROCESS OF GTL

(75) Inventors: Kazuzumi Tomiyoshi, Kawasaki-shi (JP); Masakatsu Hirano, Komogawa-shi (JP)

(73) Assignee: ASIAN CLEAN ENERGY PRIVATE LIMITED, Singapore (SG)

(21) Appl. No.: 13/508,635

(22) PCT Filed: Mar. 30, 2010
(86) PCT No.: PCT/JP2010/055750

§ 371 Date: May 8, 2012

(30) Foreign Application Priority Data
Nov. 10, 2009 (JP) PCT/JP2009/069094

(51) Int. Cl. C07C 2/00

(52) U.S. Cl. 585/310

(57) ABSTRACT

There is provided a method in which straight-chair saturated hydrocarbon can be produced, at a high relative reducibility, directly from natural gas in a direct process of GTL by using a single master oil. Electromagnetic wave is irradiated to resolve natural gas thereby to produce methylene (CH₂) which is mixed with the straight-chain saturated hydrocarbon having carbon elements of 5 to 30 to unite methylene with each other so as to have the same number of the carbon elements as that of the carbon elements of the straight-chain saturated hydrocarbon.

### TECHNICAL FIELD

This invention relates to a method of producing straight-chain saturated hydrocarbon in which it can be produced, at a high relative reducibility, from natural gas in the direct process of GTL (gas to liquid) without performing F-T synthesis.

### BACKGROUND OF THE INVENTION

Natural gas is a clean energy which has less influence on environmental pollution in comparison with other fossil fuels and people pay attention to it as an important energy. However, since the natural gas is a gaseous body, it has problems of transportation and storage. Therefore, in the development of a gas field, the scale of the gas field, the distance of transportation and the desire of users must be suitable for liquefaction (LNG) and transportation by pipelines.

GTL (gas to liquid) is known as a technology for producing hydrocarbon from natural gas, and it is expected as a technology with respect to an undeveloped gas field. At present, the technology of GTL performed in the industries is a chemical indirect method which is called F-T synthesis. That is, natural gas is transformed into synthetic gas (synthesis gas production process) by synthesizing the natural gas and stream at a high pressure, the synthetic gas is brought into contact with iron catalyst (F-T reaction) to combine carbon elements with each other (F-T synthetic process) to unite them in the shape of chain, a long chain of carbon elements are cut into a predetermined length by hydrogenation (hydrogenation resolving process), and pieces of chains are classified by distillation in accordance with their lengths to produce synthetic oil as a substitute for fuel oil.

The synthetic oil obtained in this system includes only a small amount of sulphur to decrease environmental pollution without a diesel engine and gasoline. Further, the development of this technology has been performed by many technologists.

Patent Document 1: Japanese Laid Open Publication 2006-263614

### SUMMARY OF THE INVENTION

### Problem to be Solved by the Invention

However, the GTL technology using the F-T synthesis includes many production processes and needs much initial cost for plant investment and much working fund in order to industrialize it. Further, since the F-T synthesis is performed at a pressure of 2 MPa to 5 MPa, there may be an explosion accident and the relative reducibility from the natural gas is a low ratio of 35% to 40%. The relative reducibility means the ratio at which natural gas of 1 m³ is liquefied. In addition, the products produced by means of the F-T synthesis is not a single kind of oil and its usage is limited.

It is an object to provide a method of producing straight-chain saturated hydrocarbon in the direct process of GTL in which the straight-chain saturated hydrocarbon can be produced directly from the natural gas at a high rate of relative reducibility without a process in which the natural gas is transformed into synthetic gas.

### Means for Solving the Problem

The characteristic feature of this invention resides in that a method of producing straight chain saturated hydrocarbon in direct process for GTL, which comprises:

a methylene production process for irradiating electromagnetic wave to natural gas to resolve it thereby to produce methylene (CH₂); and a copying process for mixing the methylene produced in the methylene production process with first straight-chain saturated hydrocarbon as shown by Formula 1 mentioned below to unite methylene elements with each other to produce second straight-chain saturated hydrocarbon so that the number of carbon elements of the first straight-chain saturated hydrocarbon is the same as that of carbon elements of the second straight-chain saturated hydrocarbon.

CₙH₂ₙ₊₂ (n=5 to 30) Formula 1

In the copying process, the methylene elements (CH₂) may be united with each other in the copying method in accordance with natural frequency of the straight-chain saturated hydrocarbon.

In the copying process, the straight-chain saturated hydrocarbon shown by the Formula 1 may be mixed, in the shape of mist, with the methylene (CH₂) produced in the methylene production process.

In the copying process, there may be provided a mixing vessel having an outer cylinder with a bottom wall and an inner cylinder disposed at a position separated from the bottom wall of the outer cylinder, gas is supplied between the outer and inner cylinders while revolving the gas therebetween so as to go upwardly, and the methylene (CH₂) produced in the methylene production process and the straight-chain saturated hydrocarbon shown by the Formula 1 are supplied into the mixing vessel so as to be mixed with each other while they are revolved.

The natural gas used in the methylene production process may be heated at a temperature of 180° C. to 200° C. and the electromagnetic wave irradiated in the methylene production process may has a frequency of 20 GH_{Z} to 30 GH_{Z}. Further, the natural gas used in the methylene production process may be methane (CH₄), and, in the methylene production process, the natural gas may be brought into contact with nickel (Nᵢ) catalyst.

The supplied gas may be nitrogen (N₂) or argon (Ar).

In addition, magnesium tourmaline catalyst may be accommodated in the mixing vessel and, in the copying process, the methylene supplied into the mixing vessel is mixed with the straight-chain saturated hydrocarbon after the methylene passes through the magnesium tourmaline catalyst.

The mixing vessel has magnesium tourmaline catalyst therein and, in the copying process, the methylene and the magnesium tourmaline catalyst are brought into contact with each other while the methylene is mixed with the straight-chain saturated hydrocarbon.

### Effect of the Invention

According to this invention, the straight-chain saturated hydrocarbon can be produced directly from natural gas at a high rerative reducibility without a process in which the natural gas is transformed into synthetic gas.

### BRIEF DESCRIPTION OF THE DRAWINGS

FIG. 1 is a flow chart for showing an example of a method of producing straight-chain saturated hydrocarbon according to this invention.

FIG. 2 is a schematic view for showing an example of a method of producing straight-chain saturated hydrocarbon according to this invention.

FIG. 3 is a view for showing a mechanism in which molecules of methylene are united with each other.

FIG. 4 is a schematic view for showing an example of a copying machine.

FIG. 5 is a schematic view for showing a working example.

FIG. 6 is a view for showing a nickel catalyst layer as a working example.

### MODE FOR CARRYING OUT THE INVENTION

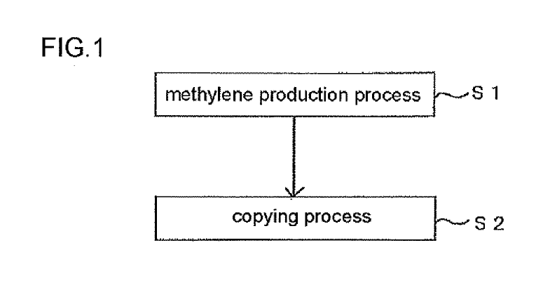

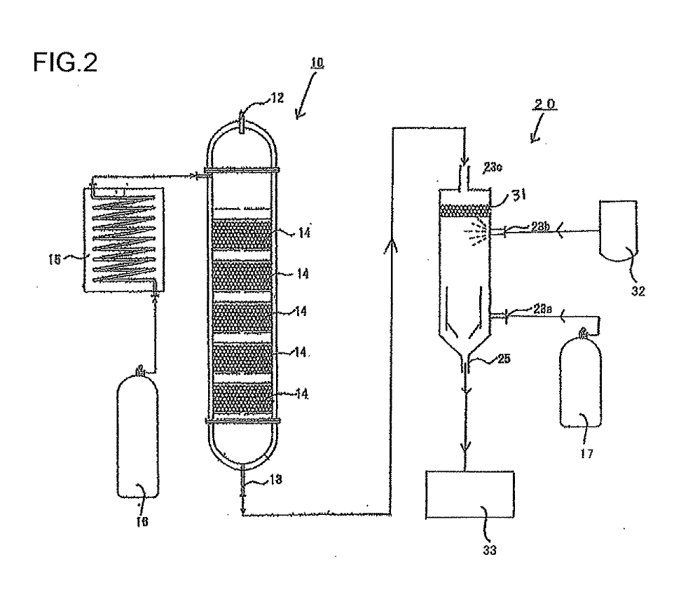

An example of the method of producing straight-chain saturated hydrocarbon according to this invention will now be concretely explained with reference to the drawings. FIG. 1 is a flow chart for showing the method of producing the straight chained saturated hydrocarbon according to this invention, and FIG. 2 is a schematic view for showing an example of the method of producing the straight chained saturated hydrocarbon.

As shown in FIG. 1, the method of producing the straight chained saturated hydrocarbon according to this invention comprises a methylene producing process in which electromagnetic wave is irradiated to resolve natural gas to produce methylene (CH₂) and a union process in which the methylene molecules produced in the methylene production process are mixed with the straight chained saturated hydrocarbon with carbon elements (molecules) of 5 to 30 as described by the following Formula (1) to unite methylene molecules with one another to make new straight chained saturated hydrocarbon with the same number of carbon elements as that of carbon elements of the mixed straight chained saturated hydrocarbon.

CₙH₂ₙ₊₂ (n=5 to 30) Formula 1

(Methylene Producing Process)

In the methylene producing process, electromagnetic wave is ejected at the natural gas to be resolved so that methylene is produced from the natural gas. More concretely, electrolysis generated by electromagnetic wave cuts molecular chains of the natural gas to produce methylene.

The methylene production process will now be concretely explained with reference to FIG. 2 which shows an example of the methylene production process. This invention is not limited to the mode shown in FIG. 2.

In FIG. 2, a methylene production apparatus has a natural gas inlet 11 for supplying continuously natural gas into a methylene production device 10, an electromagnetic wave generation device 12 for ejecting electromagnetic wave to the natural gas supplied into the methylene production device 10, a methylene outlet 13 for discharging the produced methylene, and a plurality of catalyst layers 14 through which the natural gas passes.

In this process, various kinds of natural gases can be used. For example, methane gas, ethane gas, propane gas, pentane gas, etc., can be used. Especially, methane gas is preferable. The natural gas is supplied into the methylene production device 10 through the natural gas inlet 11. In FIG. 2, the natural gas in a gas tank 16 is heated by a gas heater 15, and, thereafter, is supplied into the methylene production device 10 through the natural gas inlet 11.

The temperature of the natural gas (e.g. methane) used in this process is not particularly limited, and, however, it is preferable to use the natural gas which is heated at a temperature of 180° C. to 200° C. The natural gas with such a range of the temperature can increase the productive efficiency of the methylene gas. The method of heating the natural gas is not limited. As shown in FIG. 2, the natural gas can be heated by the gas(oil) heater 15. Further, it may be heated by a burner, etc. In addition, the natural gas is not necessarily heated before it is supplied into the methylene production device 10 through the natural gas inlet 11. That is, in order to heat the natural gas, the methylene production device 10 may be heated after the natural gas is supplied thereinto. In the case that the temperature of the natural gas is 180° C. to 200° C., the heating treatment is not necessary.

It is preferable that the ingredient of sulphur is eliminated from the natural gas supplied to the natural gas inlet 11. Sulphur may be eliminated therefrom by any means and, for example, the ingredient of sulphur in the natural gas is changed into hydrogen sulfide which is eliminated by a tourmaline catalyst device. In this manner, the sulphur can be eliminated by one of desulphurization means.

The electromagnetic wave can be properly selected in accordance with the kind of natural gases so as to resolve the natural gas thereby to produce methylene gas. The frequency of the electromagnetic wave is preferably 20 GH_{Z} to 30 GH_{Z}, more preferably 23 GH_{Z} to 26 GH_{Z}. Especially, in case that methane gas is used as the natural gas, the electromagnetic wave of the above frequency can produce efficiently methylene gas.

Any electromagnetic wave generation device 12 for ejecting electromagnetic wave can be used if it has a function for ejecting electromagnetic wave to produce methylene gas by resolving natural gas. The device 12 is fixed in the methylene production device 10 so as to eject electromagnetic wave into the inside thereof.

Further, as shown in FIG. 2, the methylene production device 10 has preferably one or more catalyst layers which are filled with catalyst for promoting the resolution of the natural gas. Especially, nickel (Ni) catalyst is preferable as the catalyst. Each catalyst layer 14 may be provided at any position where the natural gas can pass through and be brought into contact with the catalyst layer 14.

When methylene is produced in the methylene production device 10, the pressure in the device 10 is more than 1 atmospheric pressure (1 atm.). However, it is preferable that the pressure is less than 2.5 atmospheric pressures (2.5 atm.) in consideration of the risk of explosion at a high pressure.

Methylene is produced by irradiating electromagnetic wave to the natural gas which has been brought into contact with the catalyst of the catalyst layer 14 or has passed through the catalyst layer 14, and, then, is discharged from the methylene outlet 13 to be transported to a methylene inlet mentioned after.

In this process, in case that the electromagnetic wave is irradiated to the natural gas, hydrogen is generated in addition to the methylene. A part of the hydrogen produced in this process is used in a copying process mentioned after.

(Copying Process)

In copying process S2, the methylene produced in the methylene production process S1 is mixed with the straight-chain saturated hydrocarbon, shown in the Formula 1, having carbon elements of 5 to 30 thereby to combine the methylene with each other so that it has the same number of carbon elements as that of carbon elements of the straight-chain saturated hydrocarbon.

First, a mechanism for combining methylene elements with each other will now be explained in the case of the straight-chain saturated hydrocarbon having 5 carbon elements (hereinafter referred to as pentane).

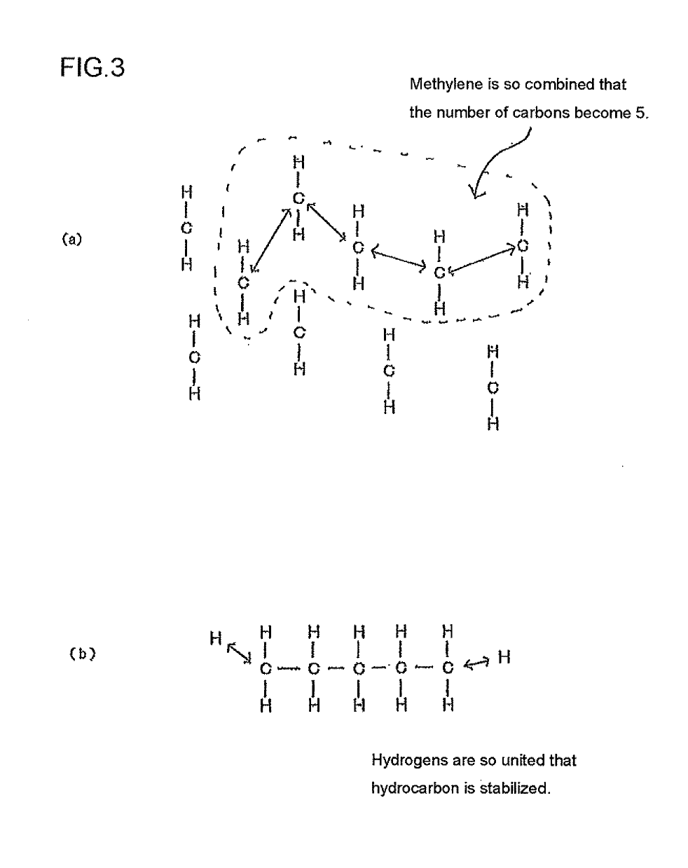

The straight-chain saturated hydrocarbon has a natural frequency in accordance with the number of carbon elements, and, when methylene is mixed with the straight-chain saturated hydrocarbon, the methylene is combined with each other in accordance with its natural frequency, in other words, so that it has the same number of carbon elements as that of carbon elements of the straight-chain saturated hydrocarbon. Therefore, when pentane is mixed with methylene as shown in FIG. 3(a), the methylene mixed with or brought into contact with the pentane is combined with each other so as to have the number of carbon elements corresponding to the natural frequency of the pentane. The state wherein methylene is combined with each other is not stable, and, therefore, the combined methylene is, as shown in FIG. 3(b) united with hydrogen produced in the methylene production process to become stable straight-chain saturated hydrocarbon. Thereby, the copying operation of the straight-chain saturated hydrocarbon (pentane) is done. That is, the straight-chain saturated hydrocarbon mixed with methylene as shown in the Formula 1 has a function as a master (seed) oil for combining methylene with each other. The straight-chain saturated hydrocarbon shown in the Formula 1 may be hereinafter referred to as a master oil.

The copied straight-chain saturated hydrocarbon is discharged from a discharging outlet 25 to be stored in a storage tank 33. A part of hydrogen produced in the methylene production process is used for a reaction in the copying process as mentioned above, and the remaining part thereof is discharged from a hydrogen outlet (not shown).

In the method of producing straight-chain saturated hydrocarbon and of having the copying process S2 according to this invention, the methylene is mixed with the master oil to produce the same straight-chain saturated hydrocarbon as the master oil. That is, if the methylene is mixed with the master oil which is desired to be produced, a desired straight-chain saturated hydrocarbon can be produced. For example, in the case that the straight-chain saturated hydrocarbon having 5 carbon elements is used as the master oil, methylene is combined with each other to produce the straight-chain saturated hydrocarbon having 5 carbon elements. If the straight-chain saturated hydrocarbon having 15 carbon elements is used as the master oil, the straight chain having 15 carbon elements can be produced. Especially, in the method of producing the straight-chain saturated hydrocarbon according to this invention, the straight-chain saturated hydrocarbon is obtained at a relative reducibility of 86% to 96% from the natural gas of 1 m³. The straight-chain saturated hydrocarbon used as the master oil may be generally gasoline having carbon elements of 5 to 11, kerosene having carbon elements of 9 to 18, light oil having carbon elements of 14 to 20 or heavy oil having carbon elements of above 17. The master oil is not necessarily a single kind of straight-chain saturated hydrocarbon. In case that a plurality of straight-chain saturated hydrocarbons having different numbers of carbon elements are used, methylene is combined with one another in accordance with a ratio of mixture to produce a mixed straight-chain saturated hydrocarbon with the ratio of mixture. For example, in case that the light oil in which straight-chain saturated hydrocarbons having 14 and 15 carbon elements, respectively, are mixed with each other at a mol ratio of 2:1 is used as the master oil, a straight-chain saturated hydrocarbon with 14 and 15 carbon elements mixed with each other at the mol ratio of 2:1 is duplicated.

The master oil is preferably in the state of mist when it is mixed with methylene. In case that the master oil in the state of mist is used, the contact area of the master oil and methylene becomes large to increase a copying efficiency. The master oil can be changed into the state of mist by means of any known method. For example, an injection nozzle may be set at a master oil inlet 23b to make the mist of the master oil.

The temperature of the master oil in the state of mist is preferably 20° C. to 50° C. in consideration of the copying efficiency of the copied straight-chain saturated hydrocarbon. In order to make the master oil with the temperature of such a range, e.g., liquid master oil may be cooled by a cooling tower (not shown).

It is preferable to mix the master oil with the methylene while they are revolved in a copying device 20 to increase the mixing efficiency. The revolving motion of the master oil and the methylene can enlarge the contacting area of them thereby to improve remarkably the copying efficiency.

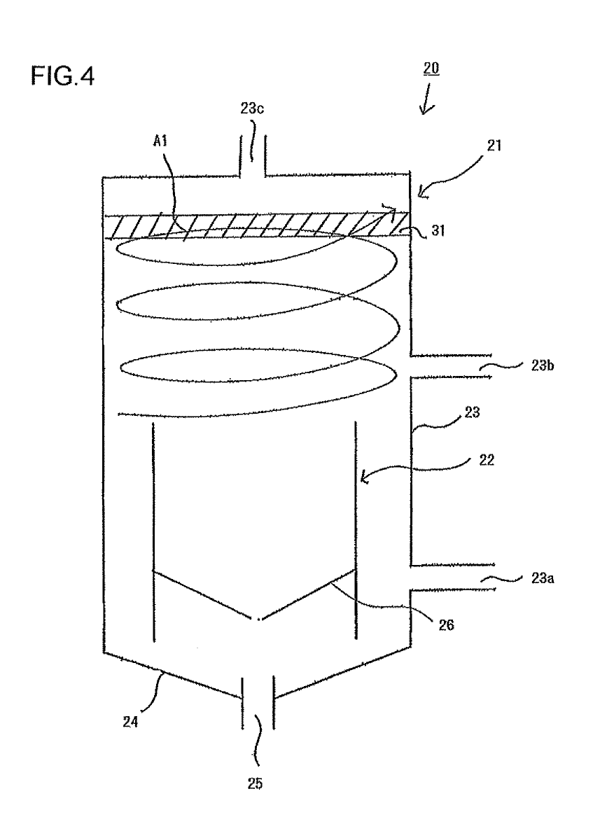

Method of mixing the methylene and the master oil while revolving them is concretely explained hereinafter with reference to FIGS. 3 and 4. FIG. 4 is a schematic view for showing an example of the copying machine.

As shown in FIG. 4, the copying machine 20 has an outer cylinder 21 and an inner cylinder 22, and the outer cylinder 21 has a cylindrical body extending vertically, a bottom wall 24 closing the bottom of the cylindrical body 23 and an upper wall closing the upper portion of the cylindrical body 23. The cylindrical body 23 has a gas inlet 23a opposed to the lower portion of the inner cylinder 20 and a master oil inlet 23b for supplying the master oil stored in a storage tank 32 into the upper portion of the inner cylinder 20. The upper wall has a methylene inlet 23c. The bottom wall 24 in the shape of a reversed cone has a discharging outlet 25 extending downwardly.

A gas inlet 23a supplies a predetermined amount of gas thereinto to generate a revolving gas current. Nitrogen (N₂) gas or argon gas which is inactive gas is preferably used as the supplied gas. Such a gas is supplied from a gas cylinder 17.

The gas supplied through the gas inlet 23a revolves between the outer and inner cylinders 21, 22 to generate a revolving gas current A1. The revolving gas current A1 revolves along the circumferential surface of the cylindrical body 23 of the outer cylinder 21 and goes upwardly in the copying machine 20.

The selected master oil which has a certain number of carbon elements of 5 to 30 is ejected in the state of mist into the copying machine 20 through the master oil inlet 23b, and the methylene produced in the methylene production process S1 is supplied into the copying machine through the methylene inlet 23c. As mentioned above, since the gas supplied into the copying machine 20 through the gas inlet 23a generates the revolving gas current A1, the master oil and the methylene are mixed with each other in the revolving gas current A1.

The methylene and the master oil are mixed to combine molecules of methylene with each other thereby to produce a straight-chain saturated hydrocarbon while copying the master oil. The copied straight-chain saturated hydrocarbon goes down into the inner cylinder 22 from the center portion of the revolving gas current to abut against a separation wall 26 which is slanted downwardly toward the center portion thereof. Therefore, the copied straight-chain saturated hydrocarbon is brought together at its center portion to be discharged from the discharging outlet 25 and is then stored in the storage tank 33.

The straight chain saturated hydrocarbon has the same properties as those of the master oil supplied from the master oil inlet 23b, and, therefore, a part of the copied straight-chain saturated hydrocarbon can be used as the master oil. For example, in the above case, if the discharging outlet or copying storage tank is connected directly or indirectly with the master oil inlet 23b or master oil storage tank 32, a part of the straight-chain saturated hydrocarbon discharged from the discharging outlet 25 can be supplied into the machine 20 through the master oil inlet 23b. In this case, new master oil is necessary for only first copying operation, and the copied hydrocarbon can be used as the master oil after the first copying operation thereby to decrease remarkably the amount of the master oil which must be prepared beforehand.

In addition, the methylene and the master oil are mixed with each other while being revolved, and, therefore, a mixing path is 3.14 times as long as a straight mixing path. For example, in the case of the copying machine 20 having the outer cylinder 21 of a diameter of 500 mm, the mixing path is 1570 mm(500×3.14). When ten revolutions are done per second, they moves for 15.7 meters per second. The revolving gas current A1 has a large centrifugal force to make the master oil in the state of mist ascend and descend repeatedly. Therefore, the master oil can be mixed sufficiently with the methylene to make relative reducibility of more than 99% possible.

Further, as shown in FIGS. 3 and 4, the copying machine 20 is preferably equipped with a catalyst layer 31 in which catalyst ejecting minus ions (e−) is accommodated. In case that the methylene is brought into contact with or made pass through the catalyst layer 31 having such a catalyst for ejecting minus ions, molecules of the methylene are easily drawn with each other to facilitate the union of each molecule of methylene. The catalyst of the catalyst layer 31 can eject minus ions to draw the molecules with each other as mentioned above, and, e.g., magnesium tourmaline catalyst can be preferably used.

The magnesium tourmaline catalyst includes a predetermined amount, e.g., 15 to 30% of magnesium in tourmaline.

The catalyst layer 31 can be disposed at any position as long as the methylene can pass through and/or is brought into contact with it, and, e.g., as shown in FIG. 4, it may be disposed at a position under the methylene inlet 23c so that the methylene can be mixed with the master oil in the revolving gas current A1 after passing through it. Further, the catalyst layer 31 can be disposed on the wall of the outer cylinder 21.

### WORKING EXAMPLE

The invention will be concretely explained with reference to working examples.

<Preparation of Natural Gas Storage Tank>

There is prepared natural gas of 110 L, having ingredients shown in the Table 1 which is filled up at an atmospheric pressure of 2.5 in a natural gas storage tank.

TABLE 1

| ingredient | mol (%) |
|---|---|
| CO₂ (carbon dioxide) | 1.92 |
| Nitrogen (N₂) | 0.36 |
| Methane | 89.65 |
| Ethane | 4.30 |
| Propane | 2.39 |
| i(iso)-Butane | 0.56 |
| n(normal)-Butane | 0.49 |
| i-Pentane | 0.14 |
| n-Pentane | 0.08 |
| Hexane | 0.10 |
| Total | 100 |

<First Cylindrical Tank>

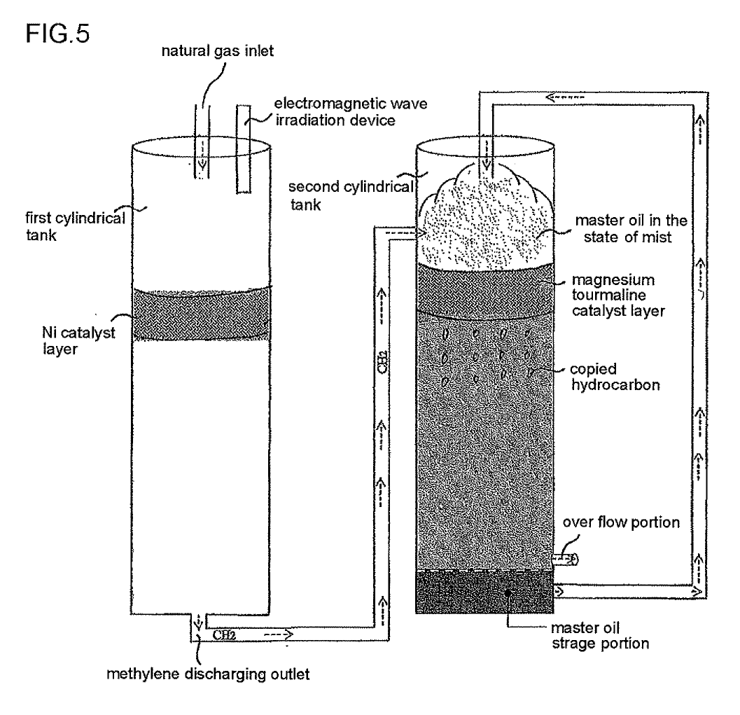

FIG. 5 shows a first cylindrical tank which was used for production of methylene according to an example of this invention. The first cylindrical tank had an inner diameter of 200 mm and a height of 200 mm, and was provided with an electromagnetic wave irradiation device and a methylene discharging outlet for discharging the methylene. A nickel catalyst layer having an inner diameter of 200 mm and a height of 100 mm was disposed at a position of 1800 mm separated from the bottom of the tank. A magnesium tourmaline catalyst layer had also the same size as that of the above catalyst layer as mentioned after.

<Production of Methylene>Before a natural gas was supplied thereinto, the first cylindrical tank was heated by a gas burner so as to maintain the temperature of the inside of the tank at 180° C. After the inside temperature reached at 180° C., the natural gas was supplied into the first cylindrical tank from the natural gas filling tank. Further, thereafter, an electromagnetic wave of 24 GH_{Z} was irradiated to the inside of the tank from an electromagnetic wave irradiation device made by KYOTO DENSHI KOGYO, LTD. until the natural gas filling tank became empty. The produced methylene was supplied into a second cylindrical tank through the methylene discharging outlet.

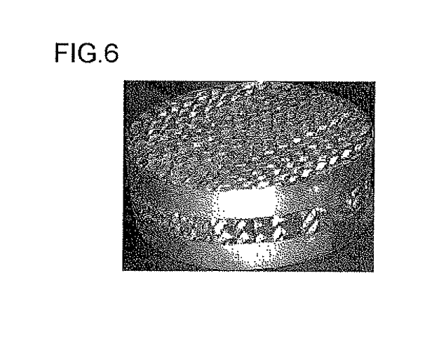

FIG. 5 shows also a second cylindrical tank which was used for copying the master oil according to the working example of this invention. The second cylindrical tank had an inner diameter of 200 mm and a height of 2000 mm. The second cylindrical tank comprised a pump for sucking up the master oil stored beforehand in the bottom portion of the tank, an injection device (diesel injection nozzle) for injecting the sucked master oil in the state of mist at the inside of the second cylindrical tank, a overflow portion for discharging the overflowing straight-chain saturated hydrocarbon copied by combining methylene molecules with each other, and a magnesium tourmaline catalyst layer having an inner diameter of 200 mm and a height of 100 mm (honeycomb structure) and positioned at a height of 1800 mm from the bottom of the second cylindrical tank (FIG. 6). The magnesium tourmaline catalyst layer included magnesium of 22%.

<Copy of Master Oil>

GTL synthetic light oil of 5 L which was produced from a natural gas and had properties shown in the Table 2 (left column) was, as a master oil, supplied into the second cylindrical tank. Thereafter, the master oil was sucked up to be injected, in the state of mist, into the upper portion of the second cylindrical tank through the diesel injection nozzle. The master oil was mixed with methylene supplied from a methylene inlet to combine methylene molecules with each other while copying the master oil. This process was continuously repeated until the overflow of the master oil was stopped, and, thus, the straight-chain saturated hydrocarbon was manufactured according to the working example.

The amount of the straight-chain saturated hydrocarbon obtained at the end of the overflow was 96 L. The properties of the hydrocarbon are shown in the Table 2.

As clearly shown in the Table 2, according to the method of producing the straight-chain saturated hydrocarbon of this invention, it is confirmed that the straight-chain saturated hydrocarbon having the same properties as the master oil has been produced and that a copying operation can be performed. Further, the hydrocarbon can be manufactured at a high rate of approximately 87%. As shown clearly in the Table 2, it is also confirmed that all properties of the hydrocarbon are better than those of the master oil.

TABLE 2

| Items of evaluation | Master oil (light oil) | Copied hydrocarbon |
|---|---|---|
| density (g/cm³) 15° C. | 0.78 | 0.7951 |
| kinetic viscosity 40° C. | 2.80 | 2.75 |
| 30° C. | 3.20 | 3.182 |
| flash point (° C.) | 88 | 80 |
| amount of sulphur (ppm) | 71 | 69 |
| cetane index | 69.5 | 80.0 |
| pour point (° C.) | under −50.0 | under −40.0 |
| clogging point (CFPP) | under −20.0 | under −10.0 |

### EXPLANATION OF NUMBER

10 . . . methylene production device

11 . . . natural gas inlet

12 . . . electromagnetic wave generation device

13 . . . methylene discharging outlet

14 . . . catalyst layer

16, 17 . . . tank

20 . . . copying machine

21 . . . outer cylinder

22 . . . inner cylinder

23 . . . side wall

24 . . . bottom wall

25 . . . discharging outlet

26 . . . partition wall

31 . . . catalyst layer

32 . . . copying storage portion

33 . . . master oil storage portion

1-11. (canceled)

12. A method of producing a straight chain saturated hydrocarbon in direct process for GTL, which comprises:
a methylene production process for irradiating electromagnetic wave to a natural gas to resolve it thereby to produce methylene (CH); and
a copying process for mixing the methylene produced in the methylene production process with a first straight-chain saturated hydrocarbon as shown by Formula 1 mentioned below to unite methylene elements with each other to produce a second straight-chain saturated hydrocarbon so that the number of carbon elements of the first straight-chain saturated hydrocarbon is the same as that of carbon elements of the second straight-chain saturated hydrocarbon.
CnH₂ₙ₊₂ (n=5 to 30) Formula 1

13. A method according to claim 12, wherein the methylene (CH₂) is united with each other in the copying method in accordance with a natural frequency of the straight-chain saturated hydrocarbon.

14. A method according to claim 12, wherein, in the copying process, the straight-chain saturated hydrocarbon shown by the Formula 1 is mixed, in the state of mist, with the methylene (CH₂) produced in the methylene production process.

15. A method according to claim 12, wherein there is provided a mixing vessel having an outer cylinder with a bottom wall and an inner cylinder disposed at a position separated from the bottom wall of the outer cylinder, a gas is supplied between the outer and inner cylinders while revolving the gas therebetween so as to go upwardly, and the methylene (CH₂) produced in the methylene production process and the straight-chain saturated hydrocarbon shown by the Formula 1 are supplied into the mixing vessel so as to be mixed with each other while they are revolved.

16. A method according to claim 12, wherein the natural gas used in the methylene production process is heated at a temperature of 180° C. to 200° C.

17. A method according to claim 12, wherein the electromagnetic wave irradiated in the methylene production process has a frequency of 20 GH_{Z} to 30 GH_{Z}.

18. A method according to claim 12, wherein the natural gas used in the methylene production process is methane (CH₄).

19. A method according to claim 12, wherein, in the methylene production process, the natural gas is brought into contact with nickel (Ni) catalyst.

20. A method according to claim 12, wherein the gas is nitrogen (N₂) or argon (Ar).

21. A method according to claim 12, wherein magnesium tourmaline catalyst is accommodated in the mixing vessel and, in the copying process, the methylene supplied into the mixing vessel is mixed with the straight-chain saturated hydrocarbon after the methylene passes through the magnesium tourmaline catalyst.

22. A method according to claim 12, wherein the mixing vessel has magnesium tourmaline catalyst therein, and, in the copying process, the methylene and the magnesium tourmaline catalyst are brought into contact with each other while the methylene is mixed with the straight-chain saturated hydrocarbon.

23. A method according to claim 13, wherein, in the copying process, the straight-chain saturated hydrocarbon shown by the Formula 1 is mixed, in the state of mist, with the methylene (CH₂) produced in the methylene production process.

24. A method according to claim 13, wherein there is provided a mixing vessel having an outer cylinder with a bottom wall and an inner cylinder disposed at a position separated from the bottom wall of the outer cylinder, a gas is supplied between the outer and inner cylinders while revolving the gas therebetween so as to go upwardly, and the methylene (CH₂) produced in the methylene production process and the straight-chain saturated hydrocarbon shown by the Formula 1 are supplied into the mixing vessel so as to be mixed with each other while they are revolved.

25. A method according to claim 14, wherein there is provided a mixing vessel having an outer cylinder with a bottom wall and an inner cylinder disposed at a position separated from the bottom wall of the outer cylinder, a gas is supplied between the outer and inner cylinders while revolving the gas therebetween so as to go upwardly, and the methylene (CH₂) produced in the methylene production process and the straight-chain saturated hydrocarbon shown by the Formula 1 are supplied into the mixing vessel so as to be mixed with each other while they are revolved.

26. A method according to claim 13, wherein the natural gas used in the methylene production process is heated at a temperature of 180° C. to 200° C.

27. A method according to claim 14, wherein the natural gas used in the methylene production process is heated at a temperature of 180° C. to 200° C.

28. A method according to claim 13, wherein the electromagnetic wave irradiated in the methylene production process has a frequency of 20 GH_{Z} to 30 GH_{Z}.

29. A method according to claim 14, wherein the electromagnetic wave irradiated in the methylene production process has a frequency of 20 GH_{Z} to 30 GH_{Z}.

30. A method according to claim 15, wherein the electromagnetic wave irradiated in the methylene production process has a frequency of 20 GH_{Z} to 30 GH_{Z}.

31. A method according to claim 16, wherein the electromagnetic wave irradiated in the methylene production process has a frequency of 20 GH_{Z} to 30 GH_{Z}.
